(19) United States
(12) Patent Application Publication
(10) Pub. No.: US 2015/0031970 A1
(43) Pub. Date: Jan. 29, 2015

(54) SYSTEMS AND METHODS FOR MONITORING OXYGEN SATURATION DURING EXERCISE

(71) Applicant: Covidien LP, Mansfield, MA (US)

(72) Inventor: David Lain, Easton, MD (US)

(73) Assignee: Covidien LP, Mansfiels, MA (US)

(21) Appl. No.: 13/952,837

(22) Filed: Jul. 29, 2013

(51) Int. Cl. A61B 5/1455; A61B 5/024; A61B 5/00; A61B 5/11

(52) U.S. Cl. CPC A61B 5/14551; A61B 5/1118; A61B 5/02416; A61B 5/742; A61B 5/6801
USPC 600/323

(57) ABSTRACT

Various systems and methods for monitoring oxygen saturation are provided. A monitoring system may include a sensor configured to obtain a physiological signal and a monitor configured to obtain the signal from the sensor and to determine a baseline oxygen saturation from the physiological signal and to generate one or more zones based at least in part on the baseline oxygen saturation. Each zone may include a different range of percentages of oxygen saturation and at least one of the zones corresponds to an anaerobic exercise condition.

### BACKGROUND

The present disclosure relates generally to monitoring devices and, more particularly, to systems and methods for monitoring oxygen saturation during exercise.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

During exercise or fitness training, athletes or their trainers often desire to monitor certain physiological characteristics of the athlete. For example, athletes may wish to receive information related to certain physiological parameters during a training session to track their performance, or athletes may wish to subsequently review certain physiological parameters measured during the training session to evaluate their performance. Athletes may also wish to design exercise programs that enable the athlete to exercise at appropriate intensities to improve their fitness or to achieve their fitness goals. In some cases, athletes may wish to design exercise programs tailored to their current fitness level. Accordingly, various devices have been developed for monitoring certain physiological characteristics of athletes during exercise. Such devices may generally provide athletes and trainers with information to analyze the athlete's performance and/or fitness level, as well as to develop exercise programs. For example, heart rate monitors are commonly used to monitor the heart rate of the athlete during exercise. Heart rate data may provide a general indication of the athlete's exercise intensity and/or the athlete's fitness level. However, heart rate data has certain limitations and may not provide information related to whether the athlete is using an aerobic or an anaerobic pathway during exercise. Thus, improved monitoring systems and techniques are needed to enable the athlete to exercise at appropriate intensities to increase the athlete's fitness level and/or to achieve the athlete's fitness goals.

### BRIEF DESCRIPTION OF THE DRAWINGS

Advantages of the disclosed techniques may become apparent upon reading the following detailed description and upon reference to the drawings in which:

FIG. 1 is a perspective view of an embodiment of a monitoring system, in accordance with an embodiment;

FIG. 2 is a block diagram of a monitoring system, in accordance with an embodiment;

FIG. 3 is an example of a graph depicting oxygen saturation and heart rate during exercise;

FIG. 4 is a method for monitoring oxygen saturation and/or heart rate during exercise, in accordance with an embodiment;

FIG. 5 is a method for generating an exercise program, in accordance with an embodiment;

FIG. 6 is a method for generating one or more zones corresponding to aerobic and/or anaerobic exercise, in accordance with an embodiment;

FIG. 7 is a method for updating an exercise program, in accordance with an embodiment; and

FIG. 8 is front view of a display of a monitoring system, in accordance with an embodiment.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

During exercise, an athlete (e.g., a subject) may surpass aerobic capacity and may incorporate anaerobic pathways to maintain or increase exercise intensity (e.g., power). Aerobic metabolism generally occurs during periods of moderate exercise intensity when there is a sufficient supply of oxygen to the athlete's muscles to generate energy (e.g., adenosine triphosphate or ATP). Anaerobic metabolism generally occurs at high exercise intensity when there is insufficient oxygen and the athlete's muscles therefore use stored ATP or creatine phosphate or metabolize glucose to produce ATP and by-products, such as lactate. These various types of exercise (e.g., aerobic exercise or anaerobic exercise) may affect the athlete's fitness level in different ways. For example, periods of anaerobic exercise may lead to improvements in aerobic performance and overall fitness. Additionally, interval training incorporating generally alternating periods of anaerobic exercise and aerobic exercise may lead to improved fitness levels. However, anaerobic exercise has not been well-defined and it can be difficult to determine whether an athlete is exercising at anaerobic levels using traditional monitoring systems and techniques.

Thus, provided herein is a monitoring system configured to monitor the athlete's oxygen saturation during exercise or fitness training. The monitoring system may additionally monitor the athlete's heart rate. In some cases, the monitoring system may be configured to determine whether the athlete is using an aerobic or anaerobic pathway based at least in part on the athlete's oxygen saturation and/or heart rate. The monitoring system may also establish one or more zones (e.g., exercise zones, training zones, target zones) that correspond to an aerobic condition and/or an anaerobic condition. In some cases, each of the one or more zones may be defined by a range of percentages of oxygen saturation and/or a range of values of heart rate, and each of the one or more zones may have an upper limit and a lower limit for oxygen saturation and/or heart rate. For example, a first zone may include an oxygen saturation range and/or a heart rate range corresponding to aerobic exercise, while a second zone may include an oxygen saturation range and/or heart rate range corresponding to anaerobic exercise.

The monitoring system may utilize the one or more zones in a variety of ways. For example, the monitoring system may compare the athlete's oxygen saturation to the one or more zones. Through such techniques, the monitoring system may determine whether the athlete's oxygen saturation indicates that the athlete is exercising in the first zone corresponding to aerobic exercise or the second zone corresponding to anaerobic exercise, for example. Additionally, the monitoring system may generate exercise programs based on the one or more zones and/or may guide the athlete through the exercise program. For example, the monitoring system may instruct the athlete to exercise within the first zone corresponding to aerobic exercise for a period of time, and the monitoring system may then instruct the athlete to increase exercise intensity to exercise within the second zone corresponding to anaerobic exercise. The monitoring system may be configured to guide the athlete through various types of exercise programs, including interval training exercise programs having generally alternating periods of aerobic and anaerobic exercise. In certain cases, the one or more zones and/or the exercise program may be tailored for an individual athlete. For example, the monitoring system may be configured to select and/or to generate appropriate zones based on various inputs, such as baseline oxygen saturation and/or heart rate data of the athlete, a user's qualitative estimation of the athlete's fitness level, the athlete's physical characteristics, and/or data from the athlete's previous training sessions. Such systems and techniques may inform the athlete as to whether the athlete is utilizing aerobic or anaerobic pathways during exercise, and may also enable the athlete to exercise at appropriate intensity to improve the athlete's fitness level and to achieve the athlete's fitness goals.

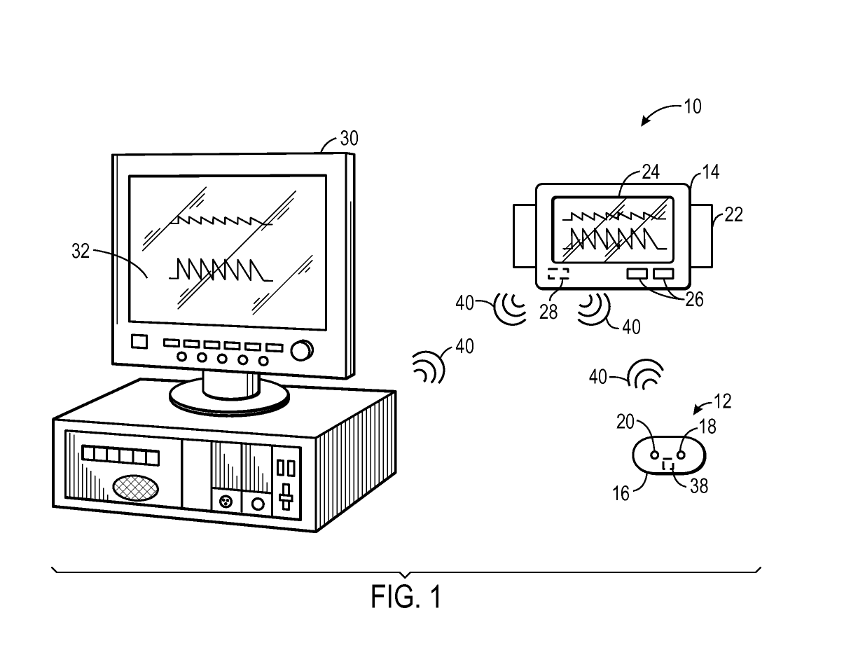

With the foregoing in mind, FIG. 1 depicts an embodiment of a monitoring system 10 that includes a sensor 12 configured to be applied to an athlete. As shown, the sensor 12 is configured to obtain a plethysmography signal, although it should be understood that any device configured to obtain oxygen saturation and/or heart rate data may be used in accordance with the techniques of the present disclosure. The system 10 may include a monitor 14 in communication with the sensor 12. The sensor 12 and the monitor 14 may communicate wirelessly as shown, or may communicate via one or more cables (e.g., the sensor 12 and the monitor 14 may be coupled via one or more cables). The sensor 12 may include a sensor body 16, which may support one or more optical components, such as one or more emitters 18 configured to emit light at certain wavelengths through a tissue of the subject and/or one or more detectors 20 configured to detect the light after it is transmitted through the tissue of the subject.

The sensor 12 or the sensor body 16 may be formed from any suitable material, including rigid or conformable materials, such as foam or other padding materials (e.g., a sponge or gel), fiber, fabric, paper, rubber or elastomeric compositions (including acrylic elastomers, polyimide, silicones, silicone rubber, celluloid, PMDS elastomer, polyurethane, polypropylene, polyethylene, acrylics, nitrile, PVC films, acetates, and latex). Furthermore, the sensor 12 or the sensor body 16 may include one or more layers (e.g., a base structural layer, an adhesive layer, and/or a foam layer). The various layers may include flexible polymeric materials (e.g., polyester, polyurethane, polypropylene, polyethylene, polyvinylchloride, acrylics, nitrile, PVC films, and acetates), foam materials (e.g., polyester foam, polyethylene foam, polyurethane foam, or the like), and adhesives (e.g., an acrylic-based adhesive, a supported transfer tape, an unsupported transfer tape, or any combination thereof). In some embodiments, the sensor body 16 may form a waterproof housing to protect the various electrical components (e.g., emitters 16, detectors 18, etc.) from moisture (e.g., sweat, rain, etc.) during use, for example.

In some embodiments, the sensor 12 may be reusable. However, in certain embodiments, the sensor 12 may be completely or partially disposable, and thus, the subject may discard all or a portion of the sensor body 12 after use. In certain embodiments, the sensor 12 may be constructed in a modular fashion such that portions of the sensor 12 (e.g., an emitter portion, a detector portion, a wireless transceiver portion, a battery portion) may be removed to be recycled into other sensors while other portions of the sensor 12 are disposed. Such a configuration may be desirable as the sensor 12 may experience wear and tear during training sessions.

The sensor 12 may be configured to be positioned on a variety of tissue locations on the subject, such as on a finger, a toe, an ankle, a wrist, or a forehead. The sensor 12 may take any suitable form to facilitate monitoring of the subject. For example, the sensor 12 may be configured to clip to or to wrap around the tissue of the subject (e.g., ear, finger, forehead, etc.). The sensor 12 may also be coupled to the subject via supportive bands or wraps attached to the sensor body 12 or that are configured to be placed over the sensor 12 and to wrap about or around a portion of the subject's body (e.g., wrap around the patient's abdomen, head, finger, etc.) to hold the sensor 12 against the subject's skin during exercise. In some embodiments, the sensor 12 may have an adhesive surface to adhere to the subject's skin or to a mounting surface of a band or a wrap.

As shown, the monitor 14 may be portable, and in some embodiments, the monitor 14 may be wearable by the patient. For example, the monitor 14 may be coupled to an attachment mechanism 22 (e.g., a strap, a clip, a band, etc.) that enables an athlete to wear the monitor 14 during exercise. Thus, the monitor 14 may be coupled to the athlete's arm, wrist, waist, or any other suitable body location. The monitor 14 may alternatively be configured to be coupled to the athlete's clothing, such as clipped onto a sleeve of a shirt, a shoelace of a shoe, or a waistband of the athlete's shorts, for example.

The monitor 14 may include a display 24 for conveying information to the athlete or user, various control devices 26 for receiving user input, a wireless module 28 for transmitting and receiving wireless data, a memory, and a processor. In certain embodiments, the physiological parameter of the patient may be calculated by the sensor 12. However, as discussed in detail below, in certain embodiments the monitor 14 may calculate the physiological parameter instead of, or in addition to, the sensor 12. Based on data received from the wireless sensor 12, the monitor 14 may display measurements and perform various measurement or processing algorithms. For example, the monitor 14 may perform blood oxygen saturation calculations, pulse rate (i.e., heart rate) measurements, and other measurements based on the data received from the sensor 12.

Furthermore, to provide additional functions and/or display options, the monitor 14 may be communicatively coupled to any number of separate portable displays and/or to a multi-parameter monitor 30, for example, via a wireless connection 40 and/or via a cable. Thus, in addition to the monitor 12, or alternatively, the multi-parameter monitor 30 may be configured to calculate physiological parameters and to provide a central display 32 for visualization of information from the monitor 14 and from other monitoring devices or systems. The multi-parameter monitor 30 may facilitate presentation of data related to the subject, such as oxygen saturation, heart rate, and information related to the subject's fitness level or training sessions. For example, the multi-parameter monitor 30 may display a graph of SpO₂ values, a current pulse rate, the one or more zones, or any other data related to the subject or the athlete's training session in a centralized location for quick reference by the athlete or other user, such as a trainer. The multi-parameter monitor 30 may be any suitable device, such as a personal computer. Any of the components of the system 10, such as the sensor 12, the monitor 14, and/or the multi-parameter monitor 30 may be configured to store data related to the athlete, the athlete's exercise programs, and/or the athlete's training sessions, and the stored data may be accessible to the athlete or to another user at any time via one of the components of the system 10 or via a network.

Like the monitor 14, the sensor 12 may include a wireless module 38. The wireless module 38 of the wireless sensor 12 may establish a wireless communication 40 with the wireless module 28 of the monitor 14 using any suitable protocol. By way of example, the wireless modules 28, 38 may be capable of communicating using the IEEE 802.15.4 standard, and may communicate, for example, using ZigBee, WirelessHART, or MiWi protocols. Additionally or alternatively, the wireless modules 28, 38 may be capable of communicating using the Bluetooth standard or one or more of the IEEE 802.11 standards. In an embodiment, the wireless module 38 may include a transmitter (such as an antenna) for transmitting wireless data, and the wireless module 28 includes a receiver (such as an antenna) for receiving wireless data. In an embodiment, the wireless module 38 also includes a receiver for receiving instructions (such as instructions to switch modes), and the wireless module 28 also includes a transmitter for sending instructions to the sensor 12.

As discussed herein, the sensor 12 may be configured to monitor a physiological parameter, such as oxygen saturation. In particular embodiments, the sensor 12 may be configured to obtain plethysmography and/or pulse oximetry data. The sensor 12 may also be configured to monitor various other physiological parameters, such as heat rate, respiration rate, tissue water fraction, hematocrit, and/or water content. The sensor 12 may include additional functionality, such as temperature or pressure sensing functionality, for example. The system 10 may also include any number of sensors and/or various different types of sensors, such as an accelerometer or a pedometer, that are configured to obtain data from the patient.

As shown in FIG. 1, the sensor 12 may include one or more emitters 18 and/or one or more detectors 20. The emitter 18 may be configured to transmit light, and the detector 20 may be configured to detect light transmitted from the emitter 18 into a patient's tissue after the light has passed through the blood perfused tissue. The detector 20 may generate a photoelectrical signal correlative to the amount of light detected. The emitter 18 may be a light emitting diode, a superluminescent light emitting diode, a laser diode or a vertical cavity surface emitting laser (VCSEL). Generally, the light passed through the tissue is selected to be of one or more wavelengths that are absorbed by the blood in an amount representative of the amount of the blood constituent present in the blood. The amount of light passed through the tissue varies in accordance with the changing amount of blood constituent and the related light absorption. For example, the light from the emitter 18 may be used to measure blood oxygen saturation, water fractions, hematocrit, or other physiological parameters of the patient. In certain embodiments, the emitter 18 may emit at least two (e.g., red and infrared) wavelengths of light. The red wavelength may be between about 600 nanometers (nm) and about 700 nm, and the IR wavelength may be between about 800 nm and about 1000 nm. However, any appropriate wavelength (e.g., green, yellow, etc.) and/or any number of wavelengths (e.g., three or more) may be used. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

The detector 20 may be an array of detector elements that may be capable of detecting light at various intensities and wavelengths. In one embodiment, light enters the detector 20 after passing through the tissue of the patient. In another embodiment, light emitted from the emitter 18 may be reflected by elements in the patent's tissue to enter the detector 20. The detector 20 may convert the received light at a given intensity, which may be directly related to the absorbance and/or reflectance of light in the tissue of the patient, into an electrical signal. That is, when more light at a certain wavelength is absorbed, less light of that wavelength is typically received from the tissue by the detector 20, and when more light at a certain wavelength is transmitted, more light of that wavelength is typically received from the tissue by the detector 20. After converting the received light to an electrical signal, the detector 20 may send the signal to the monitor 14, where physiological characteristics may be calculated based at least in part on the absorption and/or reflection of light by the tissue of the patient.

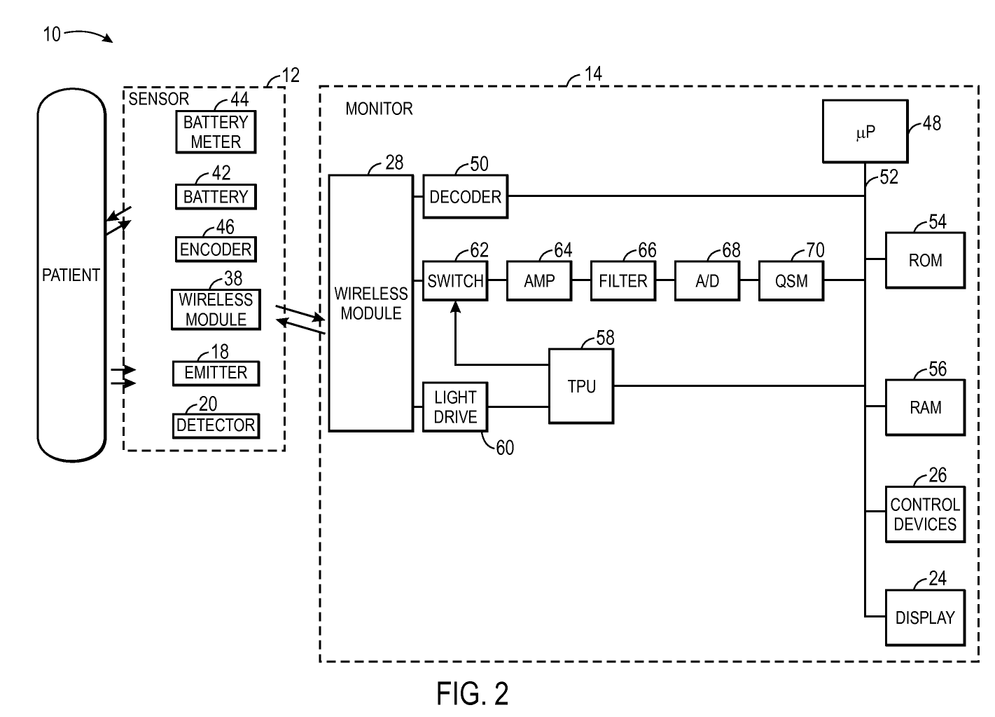

FIG. 2 depicts a block diagram of one embodiment of a patient monitoring system 10 having the sensor 12 configured to obtain physiological parameters of an athlete 40. As shown, the sensor 12 may be in wireless communication with the monitor 14. Although only one sensor 12 is depicted, two, three, four, five, or more sensors 12 may be included in the system 10. Similarly, although one monitor 14 is depicted, two, three, four, or more similar or different monitors and/or displays may be provided as part of the system 10. For example, multiple monitors 14 may be provided for use by the athlete and by the trainer.

As noted above, the sensor 12 may be configured to operate and/or to communicate wirelessly, without the use of any cables or cords. In some such embodiments, the sensor 12 may include or may be coupled to a power source 42 (e.g., a battery) to supply the sensor 12 with power. By way of example, the battery 42 may be a rechargeable battery (e.g., a lithium ion, lithium polymer, nickel-metal hydride, or nickel-cadmium battery) or may be a single-use battery such as an alkaline or lithium battery. A battery meter 44 may be included in the sensor 12 to provide the expected remaining power of the battery 42 to the athlete or to the monitor 14, for example.

The sensor 12 may also include an encoder 46 that may provide signals indicative of the wavelength of one or more light sources of the emitter 18, which may allow for selection of appropriate calibration coefficients for calculating a physical parameter such as blood oxygen saturation. The encoder 46 may, for instance, be a coded resistor, EEPROM or other coding devices (such as a capacitor, inductor, PROM, RFID, parallel resident currents, or a colorimetric indicator) that may provide a signal to a microprocessor 48 of the monitor 14 related to the characteristics of the sensor 12 to enable the microprocessor 48 to determine the appropriate calibration characteristics. In some embodiments, the encoder 46 and/or a decoder 50 may not be present.

Additionally, the sensor 12 may include or may be coupled to the wireless module 38 (e.g., wireless transceiver) to send data to the monitor 14 or to receive instructions from the monitor 14. The monitor 14 may also include the wireless module 28. Signals from the detector 20 and/or the encoder 46 may be wirelessly transmitted to the monitor 14. The monitor 14 may include one or more microprocessors 48 coupled to an internal bus 52. Also connected to the bus may be a ROM memory 54, a RAM memory 56, the display 24, and the control devices 26. A time processing unit (TPU) 58 may provide timing control signals to light drive circuitry 60, which controls when the emitter 18 is activated, and if multiple light sources are used, the multiplexed timing for the different light sources. It is envisioned that the emitters 16 may be controlled via time division multiplexing of the light sources. TPU 88 may also control the gating-in of signals from detector 20 through a switching circuit 62. These signals are sampled at the proper time, depending at least in part upon which of multiple light sources is activated, if multiple light sources are used. The received signal from the detector 20 may be passed through an amplifier 64, a low pass filter 66, and an analog-to-digital converter 68 for amplifying, filtering, and digitizing the electrical signals received from the sensor 12. The digital data may then be stored in a queued serial module (QSM) 70, for later downloading to RAM 56 as QSM 70 fills up. In an embodiment, there may be multiple parallel paths for separate amplifiers, filters, and A/D converters for multiple light wavelengths or spectra received. In one embodiment, based at least in part upon the received signals corresponding to the light received by the detectors 20, the microprocessor 48 may calculate the oxygen saturation and/or other parameters (e.g., heart rate) using various algorithms. In certain embodiments, the microprocessor 48 may calculate a pulse oximetry recovery time (PORT), which is described in more detail below. In some embodiments, the microprocessor 48 may access, select, and/or calculate one or more zones corresponding to aerobic exercise and/or anaerobic exercise using various algorithms. In certain embodiments, the microprocessor 48 may generate an exercise program based on the one or more zones using various algorithms. The algorithms used by the microprocessor 48 may use coefficients, which may be empirically determined. For example, algorithms relating to the distance between the emitter 18 and various detector elements in the detector 20 may be stored in a ROM 54 and accessed and operated according to microprocessor 48 instructions.

Furthermore, one or more functions of the monitor 14 may also be implemented directly in the sensor 12. For example, in some embodiments, the sensor 12 may include one or more processing components configured to calculate the physiological characteristics from the signals obtained from the athlete. The sensor 12 may have varying levels of processing power, and may wirelessly output data in various stages to the monitor 14. For example, in some embodiments, the data output to the monitor 14 may be analog signals, such as detected light signals (e.g., pulse oximetry signals), or processed data. Additionally, although one sensor 12 and one monitor 14 are shown in FIG. 1 and FIG. 2, it should be understood that any suitable number of sensors 12 and/or monitors 14 may be utilized to monitor the athlete. Additional monitors 14 may take any suitable form and may be in communication with the sensor 12 and/or the other monitors 14, for example. Furthermore, as previously indicated, any suitable sensor or device configured to obtain the athletes oxygen saturation, including invasive devices, may be utilized in accordance with the techniques of the present disclosure.

As indicated above, the monitoring system 10 may be configured to monitor the athlete's oxygen saturation and/or heart rate during exercise. The system 10 may also be configured to determine whether the athlete is utilizing an aerobic or an anaerobic pathway based at least in part on the athlete's oxygen saturation and/or heart rate. For example, the monitoring system 10 may compare the athlete's oxygen saturation and/or heart rate to one or more zones corresponding to various types of exercise (e.g., aerobic exercise and anaerobic exercise) to determine whether the athlete is utilizing the aerobic or the anaerobic pathways. Each of the one or more zones may be defined by a percentage or a range of percentages of oxygen saturation and/or a value or a range of values of heart rate, and each of the one or more zones may have an upper limit and a lower limit for oxygen saturation and/or heart rate. For example, a first zone may include an oxygen saturation range and/or a heart rate range corresponding to aerobic exercise, while a second zone may include an oxygen saturation range and/or heart rate range corresponding to anaerobic exercise.

The one or more zones may be generated in any of a variety of ways. For example, the one or more zones may be predetermined based on empirical data and may be stored within a memory of the monitor 14, or the one or more zones may be generated by the monitor 14 based on information related to the athlete (e.g., baseline data, user input, athlete characteristics, etc.), as discussed in more detail below. In some cases, the monitor 14 may develop an exercise program based at least in part on the one or more zones and/or may guide the athlete through the exercise program. For example, the monitor 14 may develop an interval training program and/or may prompt the athlete to adjust exercise intensity to generally alternate between a first zone corresponding to aerobic exercise and a second zone corresponding to anaerobic exercise, as described further below.

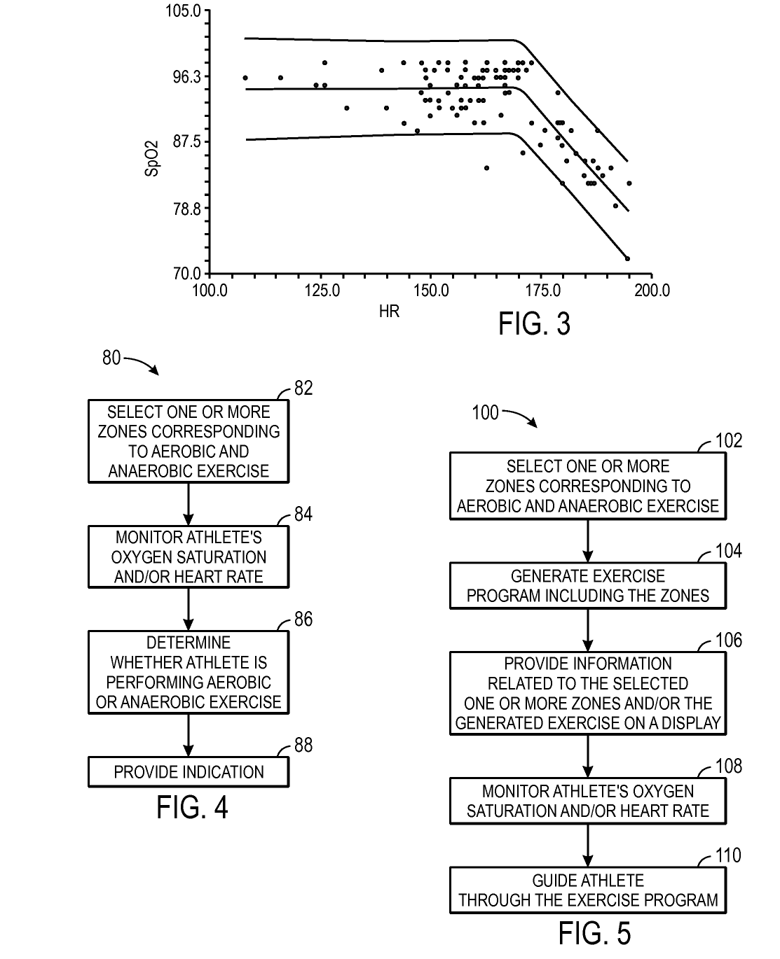

With the foregoing in mind, FIG. 3 is an example of a graph illustrating oxygen saturation data and heart rate data obtained from multiple subjects during exercise. The data in FIG. 3 was experimentally determined by monitoring the oxygen saturation and heart rate of ten elite cyclists during a training session. As each athlete's exercise intensity increases, the athlete's heart rate generally increases and the athlete's oxygen saturation generally decreases (e.g., exercise-induced arterial hypoxemia). Additionally, cluster analysis reveals a clustering of oxygen saturation and heart rate as the workload or exercise intensity increases over time. With respect to the data in FIG. 3, two distinct cluster means or averages can be identified. A first cluster of data has an average oxygen saturation of about 95% and an average heart rate of about 156 beats per minute (bpm), while a second cluster of data has an oxygen saturation of about 85% and an average heart rate of about 183 bpm. Such information can be utilized to determine or to generate one or more zones corresponding to various types of exercise. For example, the data collected in the above-described experiment and represented in FIG. 3 may be used to establish one or more zones corresponding to an aerobic condition and an anaerobic condition. In certain embodiments, each of the one or more zones includes a different oxygen saturation range and/or a different heart rate range (e.g., the ranges of the first zone do not overlap with the ranges of the second zone).

For example, as set forth in Table 1, a first zone (e.g., Zone 1) may correspond generally to an aerobic condition, while the second zone (e.g., Zone 2) may correspond generally to an anaerobic condition. Zone 1 may include an oxygen saturation value of about 95% (e.g., about 90-100%, 91-99%, 92-98%, 93-97%, 94-96%) and a heart rate of about 156 bpm (e.g., about 142-170 bpm, 150-162 bpm, 151-160 bpm, 152-159 bpm, 153-158 bpm, 154-157 bpm), while Zone 2 may include an oxygen saturation value of about 84% (e.g., about 80-88%, 81-87%, 82-86%, 83-85%) and a heart rate value of about 183 bpm (e.g., about 176-190 bpm, 177-189 bpm, 178-188 bpm, 179-187 bpm, 180-186 bpm, 181-185 bpm, 182-184 bpm).

TABLE 1

| Variable | Zone 1 | Zone 2 |
|---|---|---|
| Oxygen Saturation | 95 | 84 |
| Heart Rate | 156 | 183 |

Although only two zones are shown in Table 1, it should be understood that multiple different zones may be provided. For example, four different zones corresponding generally to a low aerobic condition, a high aerobic condition, a low anaerobic condition, and a high aerobic condition may be provided. Furthermore, the zones may include an oxygen saturation percentage and/or heart rate value, or the zones may include a range of oxygen saturation percentages and/or a range of heart rate values. In some embodiments, Zone 1 may include an oxygen saturation range of about 92-98% and a heart rate range of about 142-170 bpm, while Zone 2 may include an oxygen saturation range of about 80-88% and a heart rate range of about 176-190 bpm.

The monitor 14 may utilize the one or more zones to monitor the athlete's performance and/or to provide information to the athlete during the training session. In some embodiments, the monitor 14 may compare received oxygen saturation and/or heart rate data to the one or more zones to determine whether the athlete is performing aerobic exercise or anaerobic exercise. For example, the monitor 14 may utilize the Zone 1 and the Zone 2 set forth in Table 1, and if the athlete's oxygen saturation is approximately 95% and the athlete's heart rate is approximately 156 bpm, the monitor 14 may determine that the athlete is exercising in Zone 1 and is performing aerobic exercise.

As indicated in FIG. 3 and Table 1, the one or more zones may be based on empirical data. Thus, in some embodiments, the one or more zones may be preprogrammed (e.g., programmed at a manufacturing stage) and/or stored in a memory of the sensor 12 or the monitor 14. The monitor 14 may be configured to access the one or more zones and to use the one or more zones to determine whether the athlete is using the aerobic or anaerobic pathway, to provide information to the athlete, and/or to generate an exercise program for the athlete, as described in more detail below. However, as the zones of Table 1 were derived based on data of elite cyclists, the zones set forth in Table 1 may be most suitable for elite athletes or for athletes with a relatively high anaerobic threshold, for example. Other zones (e.g., zones having different values or ranges of oxygen saturation and/or heart rate) may be most suitable for less fit athletes or for athletes having different physical characteristics, for example. Thus, in some embodiments, the monitor 14 may store and/or access various appropriate zones based on empirical data collected from different types of athletes. Through experimentation, oxygen saturation and/or heart rate data for athlete's of various fitness levels (e.g., low, medium, high) or for athlete's having various physical characteristics (e.g., age, weight, body-mass index, gender, etc.) may be monitored during exercise sessions. The experimental data may, in turn, be used to determine various appropriate zones corresponding to aerobic exercise and anaerobic exercise for the athlete's of various fitness levels and/or for athlete's having various physical characteristics. The various appropriate zones may be preprogrammed and/or may be stored in the monitor 14 (or other suitable location). In such embodiments, the monitor 14 may be configured to access and/or to select appropriate zones for the athlete's training session. Additionally, in some embodiments, the empirically determined zones may be modified or adapted for a particular athlete. In yet other embodiments, the one or more zones may generated for the particular athlete (e.g., the zones may be based on the athlete's baseline data), for example.

FIGS. 4-7 are flow charts illustrating various methods for monitoring oxygen saturation and/or heart during exercise. The methods include various steps represented by blocks. It should be noted that any of the methods provided herein may be performed as an automated procedure by a system, such as system 10. Although the flow charts illustrate the steps in a certain sequence, it should be understood that the steps may be performed in any suitable order and certain steps may be carried out simultaneously, where appropriate. Further, certain steps or portions of the methods may be performed by separate devices. For example, a first portion of the method may be performed by the sensor 12, while a second portion of the method may be performed by the monitor 14. In addition, insofar as steps of the methods disclosed herein are applied to the received signals, it should be understood that the received signals may be raw signals or processed signals. That is, the methods may be applied to an output of the received signals.

FIG. 4 provides an example of a method for monitoring an athlete during exercise, in accordance with one embodiment. The method is generally indicated by number 80. In certain embodiments, the method 80 begins by accessing and/or selecting one or more zones corresponding to aerobic and/or anaerobic exercise in step 82. For example, the processor may access one or more zones stored in the memory of the monitor 14, in the encoder 46 of the sensor 12, or stored on a network accessible by the processor. As discussed above, in some embodiments, the one or more zones may be empirically determined and each of the one or more zones may be defined by a percentage or a range of percentages of oxygen saturation and/or a value or a range of values of heart rate. For example, a first zone may include an oxygen saturation range and/or a heart rate range corresponding to aerobic exercise, while a second zone may include an oxygen saturation range and/or heart rate range corresponding to anaerobic exercise. In some embodiments, the first zone and the second zone provided in Table 1 may be stored and made available for access by the monitor 14.

In certain embodiments, the monitor 14 may include a variety of zones stored in a database or a library, and each of the various zones may be appropriate for athletes of different fitness levels and/or different physical characteristics, for example. In such cases, the monitor 14 may access the one or more zones and may select the appropriate zones based on information related to the athlete. Such information may include any suitable information, such as baseline oxygen saturation and/or heart rate data, a user input indicating the athlete's fitness level, and/or a user input indicating the physical characteristics such as height, weight, body mass index, gender, age, and the like. At step 84, the monitor 14 may monitor the athlete's oxygen saturation and/or heart rate during exercise. In some embodiments, the monitor 14 may receive one or more signals (e.g., plethysmography signals) from the sensor 12, and the monitor 14 may process the signals to determine the athlete's oxygen saturation and/or heart rate. At step 86, the monitor 14 may determine whether the athlete is performing aerobic or anaerobic exercise. In some embodiments, such a determination may made by comparing the athlete's oxygen saturation and/or heart rate data to the one or more zones. At step 88, the monitor 14 may provide an indication of whether the athlete is performing aerobic or anaerobic exercise. The indication may be any suitable visual or audible indication, such as a written message, an audible alarm, or a colored light, for example. The indication may be provided by a speaker or by the display 24 of the monitor 14, or on any additional portable display or on the multi-parameter monitor 28, for example.

FIG. 5 illustrates a method for generating an exercise program based on one or more zones corresponding to aerobic and/or anaerobic exercise, in accordance with an embodiment. The method is generally indicated by reference number 100. In certain embodiments, the method 100 begins with accessing and/or selecting one or more zones corresponding to aerobic and/or anaerobic exercise in step 102. The one or more zones may be accessed and/or selected in the manner described above with respect to step 82 of FIG. 4, for example. In certain embodiments, the monitor may access and/or select a first zone (e.g., Zone 1) corresponding to aerobic exercise and a second zone (e.g., Zone 2) corresponding to anaerobic exercise. At step 104, a monitoring device, such as the monitor 14, may generate an exercise program including the one or more zones (e.g., the selected zones). In some embodiments, the exercise program may include a single training session, and in other embodiments, the exercise program may include a series of training sessions that are to be completed over a period of days, weeks, months, or any suitable period of time. The exercise program may include an interval training program, which may generally instruct or signal the athlete to perform relatively high intensity, anaerobic exercise interspersed with relatively low intensity, aerobic exercise or recovery periods.

In some embodiments, as shown in step 106, the monitor 14 may provide information related to the one or more zones (e.g., the selected zones) and/or the exercise program (e.g., the generated exercise program) to the athlete. For example, the monitor 14 may display a table or a chart (e.g., such as Table 1) showing the oxygen saturation range and/or the heart rate range for each of the one or more selected zones. In some embodiments, the monitor 14 may display a table or a chart showing details or parameters of the generated exercise program, such as the number of intervals, the order of the intervals, the length of the intervals, the length of the exercise program, and/or the relative intensity of the intervals. In some embodiments, the monitor 14 may provide such information to the athlete for review and/or confirmation or approval of the one or more zones and/or exercise program. In certain embodiments, the monitor 14 may enable the athlete to adjust the one or more selected zones and/or the generated exercise program. In some embodiments, the control devices 26 may enable the athlete to adjust the oxygen saturation and/or heart rate range of the one or more selected zones. For example, the athlete may desire to increase the intensity of workout, and thus, the athlete may provide input to lower the oxygen saturation range and/or to increase the heart rate range for one or more of the zones. Additionally or alternatively, in some embodiments, the control devices 26 may enable the athlete to change various parameters of the generated exercise program. For example, the athlete may wish to increase or decrease the difficulty of the exercise program by changing the length of the exercise program, the length of certain intervals, the order of the intervals, the recovery time, or the like.

At step 108, the system 10 may monitor the athlete's oxygen saturation and/or heart rate during exercise. At step 110, the system 10 may guide the athlete through the exercise program. In some embodiments, the monitor 14 may provide an indication of whether the athlete is performing within the zone prescribed by the exercise program, and the monitor 14 may provide instructions or may prompt the athlete to change exercise intensity or exercise conditions to perform within the zones prescribed for in the exercise program. For example, the exercise program may prescribe or signal the athlete to generally alternate between a first zone corresponding to aerobic exercise and a second zone corresponding to anaerobic exercise. Thus, the monitor 14 may instruct the athlete to perform within the first zone and may provide an indication of whether the oxygen saturation and/or heart rate indicate that the athlete is performing with the first zone. After a predetermined period of time or after certain oxygen saturation and/or heart rate thresholds are reached, for example, the monitor 14 may instruct the athlete to increase exercise intensity (or otherwise change exercise conditions, such as reducing available oxygen) to perform within the second zone corresponding to anaerobic exercise. The monitor 14 may provide an indication that the athlete is exercising in the second zone corresponding to anaerobic exercise when it is determined that the athlete's oxygen saturation and/or heart rate are within the second zone. After a predetermined period of time or after certain oxygen saturation and/or heart rate thresholds are reached, for example, the monitor 14 may instruct the athlete to decrease exercise intensity (or otherwise change exercise conditions, such as increasing available oxygen) to perform within the first zone corresponding to aerobic exercise. Again, the monitor 14 may provide an indication that the athlete is exercising in the second zone corresponding to anaerobic exercise when it is determined that the athlete's oxygen saturation and/or heart rate are within the first zone. The monitor 14 may continue to provide instructions in this manner to guide the athlete through the generated exercise program including the one or more zones. Additionally, in some embodiments, the monitor 14 may be configured to prompt the athlete to increase or decrease exercise intensity based on predetermined time intervals. In some embodiments, the monitor 14 may be configured to prompt the athlete to increase or decrease exercise intensity when certain oxygen saturation and/or heart rate thresholds are reached or after certain oxygen saturation and/or heart rates are achieved and maintained for a predetermined period of time.

FIG. 6 illustrates a method for generating one or more zones corresponding to aerobic and/or anaerobic exercise, in accordance with an embodiment. The method is generally indicated by reference number 120. In certain embodiments, the method 120 begins with obtaining the athlete's baseline oxygen saturation and/or heart rate (e.g., baseline cardiorespiratory data) at step 122. The baseline oxygen saturation and/or heart rate may be input and/or received at a monitoring device, such as the monitor 14. In some embodiments, the athlete's baseline oxygen saturation and/or heart rate may be determined by the monitor 14 based on a signal (e.g., physiological signal, plethysmography signal, etc.) obtained from the sensor 12, although the baseline oxygen saturation and/or heart rate may be obtained in any suitable manner. In some circumstances, the baseline oxygen saturation and/or heart rate may be obtained while the subject is resting or performing a relatively low intensity aerobic exercise for a short predetermined period of time (e.g., walking, jogging, or biking for 1, 2, 3, 4, 5, or more minutes). The baseline oxygen saturation and/or heart rate may be utilized to generate a baseline zone (e.g., Zone 0) corresponding to the athlete's baseline performance or effort.

At step 124, the monitor 14 may generate at least one zone that corresponds to an aerobic condition and at least one zone that corresponds to an anaerobic condition using one or more algorithms. In some embodiments, a first zone (e.g., Zone 1) that corresponds to an aerobic exercise condition and a second zone (e.g., Zone 2) that corresponds to an anaerobic condition may be generated based at least in part on the subject's baseline received at step 122. In certain embodiments, the first zone and the second zone may be determined based on a standard deviation of the baseline oxygen saturation and/or heart rate data. For example, the first zone may include oxygen saturation values and/or heart rate values that are within one standard deviation of the baseline oxygen saturation and/or heart rate data, while the second zone may include may include oxygen saturation values and/or heart rate values that are within two standard deviations of the baseline oxygen saturation and/or heart rate data. As noted above, the first zone and the second zone may include different ranges or values (e.g., the ranges or values do not overlap). Additionally, in some embodiments, the monitor 14 may be configured to receive and/or to consider other inputs to facilitate generation of the at least one zone. For example, information about the athlete (e.g., age, gender, weight, body mass index, etc.) may be input into the monitor 14 by control devices 26 or through any other suitable means. In some embodiments, the monitor 14 may be configured to access and/or to consider information or data from the athlete's previous training sessions, such as the athlete's oxygen saturation and/or heart rate, whether the athlete was able to perform within each of the high intensity anaerobic intervals (e.g., success rate), and/or the athlete's recovery time, which is discussed in more detail below.

At step 126, the monitor 14 may generate an exercise program including the zones established at step 124. The monitor 14 may generate the exercise program in a similar manners as set forth above with respect to step 104 of FIG. 5. For example, the monitor 14 may generate an interval training exercise program including generally alternating periods of aerobic and anaerobic exercise. Additionally, in step 128, the monitor 14 may provide information related to the established zones and/or the generated exercise program to the athlete as set forth in step 106 of FIG. 5. As set forth above, the athlete or user may adjust the zones and/or the exercise program based on personal preferences or other variables using the control devices 26, or other suitable mechanism.

At step 130, the monitor 14 may monitor the athlete's oxygen saturation and/or heart rate during the training session. In some embodiments, the monitor 14 may compare the athlete's oxygen saturation and/or heart rate to the first zone and/or the second zone. Such a comparison may enable the monitor 14 to determine whether the athlete is performing aerobic exercise or anaerobic exercise. At step 132, the monitor 14 may guide the athlete through the exercise protocol as set forth above in step 110 of FIG. 5, for example.

In some embodiments, at step 134, the monitor 14 may determine a pulse oximetry recovery time (PORT). The PORT may relate generally to the amount of time required for the athlete's oxygen saturation and/or heart rate to return to the baseline zone from the zone corresponding to anaerobic exercise (e.g., the second zone). In some embodiments, the PORT may relate generally to the amount time required for the athlete's oxygen saturation and/or heart rate to return to the zone corresponding to aerobic exercise (e.g., the first zone). In certain embodiments, the system may monitor both the amount of time required for the athlete to return to the baseline zone from relatively higher intensity zones (e.g., the first zone, the second zone) and the amount of time required for the athlete to return to the first zone from relatively high intensity zones (e.g., the second zone). The PORT may provide an indication of the athlete's ability to resynthesize creatine phosphate, which depends at least in part on the athlete's endurance level and fitness. Thus, the PORT may provide information related to the athlete's overall fitness level, and it may be desirable to provide the PORT to the athlete and/or to track the PORT during the training session. In some cases, the PORT may be used by the monitor 14 to update the zones and/or the exercise program, as described further below. For example, if the athlete's PORT is below a predetermined threshold or improves (e.g., reduces) over time, the monitor 14 may increase the intensity of the zones and/or the exercise program to be appropriate for the athlete's current fitness level. In certain embodiments, the PORT may be stored within the monitor 14 or at another suitable location on the network or in the sensor 12, for example. Thus, the PORT may be used as a baseline for later comparison to track the athlete's fitness or progress over time.

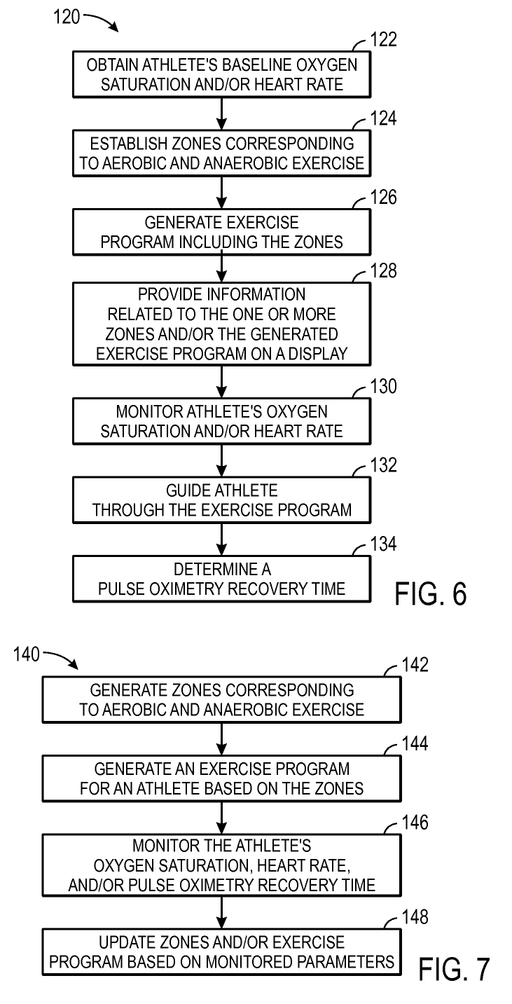

FIG. 7 provides an example of a method for updating an exercise program, in accordance with one embodiment. The method is generally indicated by reference number 140. As shown in step 142, the monitor 14 may generate or provide zones corresponding to aerobic exercise and anaerobic exercise. The zones may be accessed, selected, and/or generated via any suitable techniques, including the techniques described herein. At step 144, the monitor 14 may generate or provide an exercise program including the zones. In some embodiments, the exercise program may include one or more zones corresponding to aerobic exercise and one or more zones corresponding to anaerobic exercise. At step 146, the monitor 14 may monitor the athlete's oxygen saturation and/or heart rate during a training session. For example, the monitor 14 may receive and process a plethysmography signal to determine the athlete's oxygen saturation and/or heart rate. In some embodiments, the monitor 14 may monitor the athlete's PORT. As shown at step 148, the monitor 14 may be configured to update the zones (e.g., the oxygen saturation and/or heart rate values or ranges of each zone) and/or the exercise program. The monitor 14 may update the zones and/or the exercise program based on feedback from the oxygen saturation, heart rate, PORT, and/or any other parameters that may be monitored via the sensor 12. For example, in some embodiments, the signals generated by the sensor 12 may be processed (e.g., by the monitor 14) which may in turn evaluate the processed signals in accordance with one or more algorithms, and the monitor 14 may adjust the zones and/or the exercise program as appropriate for the athlete. In some embodiments, a learning-based algorithm or other suitable algorithms (e.g., neural networks, genetic algorithms, etc.) may be employed to evaluate the processed signal and to make adjustments to the zones and/or the exercise program. In some embodiments, the adjustments may be made during the training session (e.g., continuously or at predetermined intervals during the training session).

By way of example, if the athlete's PORT is shorter than a predetermined threshold value, the monitor 14 may adjust the exercise program to generally increase the intensity of the workout and to generally increase the demands on the athlete. Thus, the monitor 14 may adjust the second zone to have a lower oxygen saturation value and/or a higher heart rate value. Additionally or alternatively, the monitor 14 may adjust the exercise program to increase a period of time for the second zone and/or to provide a shorter recovery period (e.g., signal for the athlete to exercise within the first zone or at a baseline level for a shorter period of time before increasing intensity to exercise within the second zone). Such techniques may enable the monitor 14 to provide updated exercise programs that are appropriate for the athlete's current fitness level.

In some embodiments, the monitor 14 may additionally or alternatively be configured to update the zones and/or the exercise program based on information from the athlete's prior training session. For example, the monitor 14 may be configured to access data from the athlete's prior training session (e.g., data stored in the sensor 12, the monitor 14, or on a network) to update the zones and/or the exercise program for a current or future training session. Thus, the monitor 14 may generate or provide appropriate zones and/or exercise programs that are suitable for the athlete without requiring baseline data prior to each training session. The monitor 14 may update the zones and/or the exercise program for the athlete based on data obtained during prior training sessions as the athlete's fitness improves over time. Again in some embodiments, a learning-based algorithm or other suitable algorithms (e.g., neural networks, genetic algorithms, etc.) may be employed to evaluate the data from the prior training session and to make adjustments to the zones and/or the exercise program.

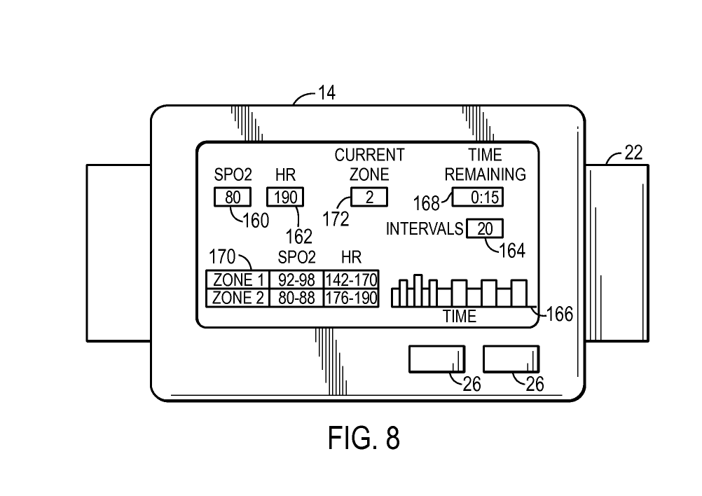

FIG. 8 provides an example of the monitor 14, in accordance with an embodiment. As shown, the monitor 14 may include the attachment mechanism to enable the athlete to wear or carry the monitor 14. Additionally, the monitor 14 may include the display 24 that is configured to provide information to the athlete or to another user, such as a trainer or coach. As shown, the display 24 provides an oxygen saturation indicator 160 and a heart rate indicator 162. The oxygen saturation and/or the heart rate may be depicted as a numerical value that is constantly updated or that is updated at suitable intervals (e.g., every 1, 2, 3, 4, 5, 6, 7, 8, 9, 10, or more seconds) during the training session, although in some embodiments, the oxygen saturation and/or the heart rate may be shown graphically (e.g., a trend over time). The display 24 may also provide an indication of a total number of intervals to be performed 164, an indication of a relative intensity of each interval (e.g., a bar graph or chart) of the training session 166, a time remaining in the current interval and/or a total duration of the interval 168, an oxygen saturation and/or heart rate for each zone or interval of the training session 170 (e.g., a chart or table), and/or an indication of the current zone (e.g., Zone 1, Zone 2, aerobic zone, anaerobic zone, etc.). Any other suitable information may be provided such as a time remaining in the training session and/or a total duration of the training session, the pulse oximetry recovery time (e.g., a trend, a chart, a table, or an indication of the most recent PORT), baseline information, and the like.

The display 24 may be a colored display and may be configured to indicate each zone by a different color. For example, the training session may begin with the athlete exercising within the first zone corresponding to aerobic exercise and at least a portion of the display 24 may have a first color (e.g., red, blue, green, yellow, orange, pink, etc.) indicative of the first zone. The monitor 14 may prompt the athlete to increase exercise intensity to exercise within the second zone corresponding to anaerobic exercise, and at least a portion of the display 24 may have a second color indicative of the second zone. Thus, the athlete may be able to identify the current type of exercise being performed or may be prompted to change exercise intensity by the monitor 14 by observing the color on the display 24.

In some embodiments, the monitor 14 may include control devices 26. Thus, the athlete or other user may interact with the monitor 14. For example, the monitor 14 may present a menu to the athlete, and the athlete may be able to navigate through various displays and/or may be able to view information, such as previous physiological data, a summary of the training session, and the like. The control devices 26 may enable the athlete to adjust the exercise program as needed and/or input information related to the athlete's fitness level or fitness goals, for example.

The monitoring system and methods provided herein may be used to monitor an athlete's oxygen saturation and/or heart rate during exercise. Such data may provide information that enables the athlete to exercise at appropriate intensity level. For example, the oxygen saturation and/or heart rate may provide an indication of whether the athlete is performing aerobic or anaerobic exercise. Additionally, the monitoring system may be configured to generate one or more zones that correspond to aerobic exercise and/or anaerobic exercise. The monitoring system may utilize the one or more zones to determine whether the athlete's oxygen saturation indicates that the athlete is exercising in the first zone corresponding to aerobic exercise or the second zone corresponding to anaerobic exercise, for example. Additionally, as set forth above, the monitoring system may generate exercise programs based on the one or more zones and/or may guide the athlete through the exercise program. In certain cases, the one or more zones and/or the exercise program may be tailored for an individual athlete, and in some cases, the one or more zones and/or the exercise program may be updated based on monitored parameters or other input.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims. Further, it should be understood that certain elements of the disclosed embodiments may be combined or exchanged with one another.

What is claimed is:

1. A monitoring system comprising:
a sensor configured to obtain a physiological signal;
a monitor configured to:
obtain the physiological signal from the sensor;
determine a baseline oxygen saturation from the physiological signal; and
generate one or more zones based at least in part on the baseline oxygen saturation, wherein each zone includes a different range of percentages of oxygen saturation and at least one of the zones corresponds to an anaerobic exercise condition.

2. The monitoring system of claim 1, wherein the sensor and the monitor are configured to communicate wirelessly.

3. The monitoring system of claim 1, wherein the monitor comprises a control device configured to receive a user input, and the control device enables a user to adjust the range of percentages of oxygen saturation included within at least one of the zones.

4. The monitoring system of claim 1, wherein at least one of the zones corresponds to an aerobic exercise condition.

5. The monitoring system of claim 1, wherein the monitor is configured to generate an exercise program including the one or more zones.

6. The monitoring system of claim 5, wherein the monitor is configured to guide the athlete through the exercise program.

7. The monitoring system of claim 6, wherein the exercise program includes an interval training program that alternates between the one or more zones.

8. The monitoring system of claim 1, wherein the at least one zone corresponding to the aerobic exercise condition includes oxygen saturation values that are within one standard deviation of the baseline oxygen saturation.

9. The monitoring system of claim 8, wherein the at least one zone corresponding to the anaerobic exercise condition includes oxygen saturation values that are greater than one standard deviation of the baseline oxygen saturation and are within two standard deviations of the baseline oxygen saturation.

10. A monitor comprising:
a sensor interface configured to receive a physiological signal from a sensor applied to a subject; and
a processor configured to establish at least one zone corresponding to an anaerobic exercise condition based at least in part on the physiological signal received from the sensor, wherein the at least one zone includes a range of percentages of oxygen saturation.

11. The monitor of claim 10, comprising a display configured to provide an indication of whether the subject is exercising within the at least one zone corresponding to the anaerobic exercise condition.

12. The monitor of claim 10, wherein the processor is configured to generate an exercise program including the at least one zone corresponding to the anaerobic exercise condition.

13. The monitor of claim 12, wherein the processor is configured to guide the subject through the exercise program.

14. The monitor of claim 12, wherein the processor is configured to determine a pulse oximetry recovery time.

15. The monitor of claim 14, wherein the processor is configured to update the range of percentages of oxygen saturation included within the at least one zone corresponding to the anaerobic condition based on the pulse oximetry recovery time.

16. A method comprising:
receiving, onto data processing circuitry, data obtained from a sensor applied to a subject;
determining, using the data processing circuitry, at least a first zone and a second zone based on the data obtained from the sensor, wherein the first zone corresponds to an aerobic exercise condition and the second zone corresponds to an anaerobic exercise condition;
monitoring the athlete's oxygen saturation during exercise and comparing the athlete's oxygen saturation to the first zone and to the second zone to determine whether the subject is performing aerobic or anaerobic exercise; and
providing an indication related to whether the subject is performing aerobic or anaerobic exercise.

17. The method of claim 16, comprising generating an exercise program including the first zone and the second zone.

18. The method of claim 17, comprising guiding the subject through the exercise program by prompting the subject to exercise within the first zone and the second zone.

19. The method of claim 16, wherein the first zone and the second zone each include a range of percentages of oxygen saturation different from one another.

20. The method of claim 19, comprising updating the range of percentages included in the first zone and the second zone based on the athlete's oxygen saturation during exercise.
